(19) United States
(12) Patent Application Publication
(10) Pub. No.: US 2006/0212086 A1
(43) Pub. Date: Sep. 21, 2006

(54) Gastrointestinal volume manipulation

(76) Inventors: Martin P. Mintchev, Calgary (CA); Emil Neshev, Calgary (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/378,676

(22) Filed: Mar. 17, 2006

Related U.S. Application Data
(60) Provisional application No. 60/662,346, filed on Mar. 17, 2005.

(51) Int. Cl. A61N 1/18

(52) U.S. Cl. 607/40

(57) ABSTRACT

A method of reducing gastric volume in a portion of a gastrointestinal tract having a longitudinal axis. The method comprises repeating a stimulation cycle comprising the steps of stimulating the gastrointestinal tract at a first location for a first stimulation period and stimulating the gastrointestinal tract at a second location on for a second stimulation period. In the disclosed method the first and second locations are axially spaced from each other along the longitudinal axis and the first and second stimulation periods are sufficient to maintain a gastric volume reduction in the portion of the gastrointestinal tract at least during the stimulation cycle.

### CROSS-REFERENCE TO RELATED APPLICATIONS

This application claims the benefit under 35 USC 119(e) of provisional patent application No. 60/662,346 filed Mar. 17, 2005.

### BACKGROUND

The digestive system processes ingested food and liquids to allow nutrients and other food substances to be absorbed by the body. One element of the digestive process is intestinal peristalsis, i.e. the coordinated and self-regulated motor activity of the intestinal tract which propels processed foodstuff through the gastrointestinal tract. Further, gastric emptying plays an important role in regulating the volume of food intake. Several studies have shown that gastric distention acts as a satiety signal to inhibit food intake and rapid gastric emptying is closely related to overeating and obesity.

The proximal stomach plays an important role in the accommodation of food. In order to fulfill this role, the proximal stomach is able to relax in response to different stimuli. Mid-corpus and distal stomach volumes are also important for quantity of food intake. Some studies have documented accelerated gastric emptying in obese people, which may be a result of rapid meal distribution to the antrum and ingesting a great deal of food without necessarily having a large proximal stomach. It has been suggested that larger fasting antral volumes could cause a change in the sense of satiety. It has also been demonstrated that fasting and postprandial volume of the distal stomach is greater in obese people. Furthermore, it was found that distal gastric volumes were larger than proximal volumes in obese individuals. To date, permanent gastric volume reduction has been achieved by surgical gastric volume restrictive methods such as gastric banding.

### SUMMARY

According to an aspect of the invention, there is provided a method of reducing gastric volume in a portion of a gastrointestinal tract, such as the stomach, having a longitudinal axis. The method in one aspect comprises the steps of stimulating at least a first contraction at a first location on the gastrointestinal tract for a first stimulation period, and stimulating at least a second contraction at a second location on the gastrointestinal tract for a second stimulation period. This cycle may be repeated. The first, the second and any subsequent locations are axially spaced from each other along the longitudinal axis. The first and second stimulation periods are sufficient to maintain a gastric volume reduction in the portion of the gastrointestinal tract stimulated. The overall contraction may be maintained approximately constant during a number of stimulation cycles.

Stimulating the contractions may be carried out using respective first and second sets of electrodes implanted on the stomach, which may be spaced circumferentially around the stomach and/or at different positions along the gastric axis, and may have alternating polarity. The electrodes may be implanted sub-serosally or sub-mucosally. Each of the first and second sets of electrodes may comprise a pair of electrode sub-sets, in which electrodes in each of the electrode sub-sets are spaced around the circumference of the portion of the gastrointestinal tract being stimulated. Stimulating either of the contractions may be carried out asynchronously with spontaneously existing slow waves in the portion of the gastrointestinal tract being stimulated.

The stimulation cycle may comprise applying stimulating energy at a frequency of 20 Hz or higher or at a frequency between 5 to 50,000 Hz. The stimulation cycle may comprise applying stimulating energy having an amplitude between 3 V peak-to-peak and 30 V peak-to-peak. The stimulation cycle may comprise applying stimulating energy in sessions of pulses, wherein each pulse has a duration of at least a second or greater, the delay between the first stimulation period and the second stimulation period may have a duration of at least a second or greater, and the delay between sessions of pulses applied at one location may have a duration equal to or greater than the duration of the stimulation session and not less than the duration of the session of pulses applied at the other location. The stimulation cycle may comprises applying excitation energy to the electrodes with a duty cycle having a pulse duration less than or equal to one half of the duty cycle.

According to another aspect of the invention, the stimulation is applied via neural pathways at the stimulated locations. The neural pathways at the first location have a first exhaustion period, and the neural pathways at the second location have a second exhaustion period. The first stimulation period may be less than or equal to the first exhaustion period in any one stimulation cycle, and the second stimulation period may be less than or equal to the second exhaustion period in any one stimulation cycle.

According to another aspect of the invention, the stimulation cycle further comprises stimulating a contraction at a third location on the gastrointestinal tract, for example via neural pathways at the third location, the first, second and third locations being axially spaced from each other along the gastric axis.

According to another aspect of the invention, there is provided a method of causing muscles to contract in a portion of the gastrointestinal tract having a longitudinal axis, the method comprising the steps of implanting a set of electrodes on the portion of the gastrointestinal tract, the set of electrodes being implanted to form a pair of circumferentially spaced electrode sub-sets, and energizing electrodes in the set of electrodes simultaneously to cause a contraction of smooth muscle of the portion of the gastrointestinal tract by signals applied via neural pathways in the vicinity of the electrodes, the signals being applied for a duration sufficient to reduce gastric volume in the portion of the gastrointestinal tract. The method may also comprise implanting a second set of electrodes on a second portion of the gastrointestinal tract, the second set of electrodes being implanted to form a pair of circumferentially spaced electrode sub-sets and energizing electrodes in the second set of electrodes simultaneously to cause a contraction of smooth muscle of the portion of the gastrointestinal tract by signals applied via neural pathways in the vicinity of the electrodes, the signals being applied for a duration sufficient to reduce gastric volume in the portion of the gastrointestinal tract, wherein the second set of electrodes are not energized while the first set of electrodes are energized.

### BRIEF DESCRIPTION OF THE DRAWINGS

Embodiments of the invention will be described with reference to the accompanying drawings, in which:

FIG. 1A is a schematic drawing of a human stomach with 4 circumferentially implanted electrode sets, where the electrodes are positioned with their axes parallel to the gastric axis;

FIG. 1B is a schematic drawing of a human stomach with 4 circumferentially implanted electrode sets, where the electrodes are positioned with their axes perpendicular to the gastric axis;

FIG. 2 shows two distal and two proximal electrode sets positioned around the gastric axis and illustrates the alternating ground/active electrode placement within each circumferential electrode set;

FIG. 3 shows the signal parameters and the time characteristics of signals applied to the electrode sets;

FIG. 4A shows a distal circumferential contraction in the stomach in the vicinity of the distal electrode sets where the electrodes are parallel to the gastric axis and subserosally implanted;

FIG. 4B shows a proximal circumferential contraction in the stomach in the vicinity of the proximal electrode sets where the electrodes are parallel to the gastric axis and subserosally implanted;

FIG. 5A shows a distal circumferential contraction in the stomach in the vicinity of the distal electrode sets where the electrodes are parallel to the gastric axis and submucosally implanted;

FIG. 5B shows a proximal circumferential contraction in the stomach in the vicinity of the proximal electrode sets where the electrodes are parallel to the gastric axis and submucosally implanted;

FIG. 6 shows a distributed microsystem setup; and

FIG. 7 depicts a detailed block diagram of an embodiment of a neurostimulator microsystem powered by an external abdominal belt.

### DESCRIPTION

In the claims, the word “comprising” is used in its inclusive sense and does not exclude other elements being present. The indefinite article “a” before a claim feature does not exclude more than one of the feature being present.

One embodiment of the present invention utilizes electrically-invoked non-spontaneous circumferential contractions in combination with appropriate control and timing mechanisms to achieve dynamic gastrointestinal (GI) volume manipulation. Electrically invoked non-spontaneous contractions in the vicinity of the stomach, either in fasting or postprandial states, or both, mimic the effects of mechanical gastric banding. However, the effect of gastric volume reduction is obtained in the present invention by producing circumferential contractions in various parts of the stomach, rather than having permanent mechanical banding at a particular area of the organ. The present invention may be utilized to invoke contractions in various segments of the GI tract, including the esophagus, the stomach, the small intestine, the large intestine (with or without additional stimulation of the lower esophageal sphincter), the pylorus, and the anal sphincter, or a combination thereof. It will be understood that the term “GI tract” as used herein refers to any of these parts or combinations thereof.

This present invention can either be used separately or in combination with other methods known in the art. The technique may be performed in a minimally invasive manner to effectively reduce gastric volume in either fasting or postprandial states, or both, which can be useful for the treatment of obesity or other disorders or states where GI volume manipulation is beneficiary.

In one embodiment a dynamic but long-term sustainable change in volume is achieved by applying controlled and appropriately timed electrical signals to sets of circumferentially-implanted electrodes either from the serosal or from the mucosal side of the GI organ under stimulation. Some embodiments utilize integrated and embedded programmable electronic timing mechanisms, to deliver electrical signals which induce strong and sustained circumferential contractions of the organ wall in the vicinity of the circumferentially-implanted electrodes, The circumferential contractions result in dynamically-maintained substantial change in the volume of the GI tract.

In one embodiment an implantable pulse generator provides the desired functionalities. The pulse generator can be subcutaneously implanted, placed within the abdominal cavity or positioned inside the lumen of the organ. Further the pulse generator may be either autonomously or transcutaneously powered.

At least one embodiment achieves volume manipulation by overriding the spontaneously existing gastrointestinal (GI) motility and inducing externally-invoked contractions, asynchronously with the spontaneously existing mechanical phenomena in the GI tract. In these embodiments multiple circumferential sets of electrodes are implanted either circumferentially or longitudinally along the longitudinal axis of the organ. The implantable pulse generator delivers precisely controlled electrical signals so as to produce powerful simultaneous contractions in the vicinity of two or more circumferential sets of electrodes, dynamically and in alternating locations, so that the net effect of the invoked contractions is substantial gastric volume reduction.

The subserosally- or submucosally-implanted electrodes provide, by way of example, externally-invoked synchronized electrical signals to the smooth muscles of the GI tract via the neural pathways of that smooth muscle. The external signals (i.e. non-intrinsic signals) supplied to the electrode sets, although synchronized between themselves, are asynchronous with the spontaneously existing slow waves in the particular GI organ, and override them, rather than stimulating or enhancing the existing intrinsic electrical activity.

The present invention may be implemented using different combinations of circumferential sets of electrodes to achieve this dynamic volume reduction. One embodiment utilizes a first circumferential set of electrodes implanted in a distal portion of the stomach and a second set of electrodes implanted in a proximal portion of the stomach. In this embodiment a circumferential contraction is produced in the distal part of the stomach. After this contraction is sustained for a given period of time, the implantable pulse generator terminates stimulation to the distal electrode set and delivers stimulation to the proximal set of electrodes (located, for example, in the mid corpus) to achieve approximately the same percentage of gastric volume reduction in the proximal area.

Subsequently, after an appropriate recovery time for the distal portion of the organ, the proximally produced contraction is relaxed, and the distally-located contraction is invoked again, thus dynamically achieving the net effect of permanent gastric volume reduction without the use of gastric banding. In one embodiment the sites of the invoked contractions are alternated between distal and proximal channels because sustained stimulation at a particular electrode location exhausts acetylcholine (ACh) secretion in the vicinity of the circumferential sets of electrodes where an invoked contraction is induced. In some embodiments each contraction engages a substantial circumferential area of the gastric wall, about 4-5 cm wide. Alternatively, other embodiments simultaneously stimulate two or more electrode sets to induce a combined contraction having a scope on the order of 10 cm or more, which further reduces the gastric volume in the stimulated organ.

Various embodiments can therefore provide minimally invasive treatment for gastrointestinal abnormalities that is customizable to each patient and completely reversible. Electrode and microsystem device implantation can be performed surgically or laparoscopically.

Simultaneously Invoked Circumferential Contractions

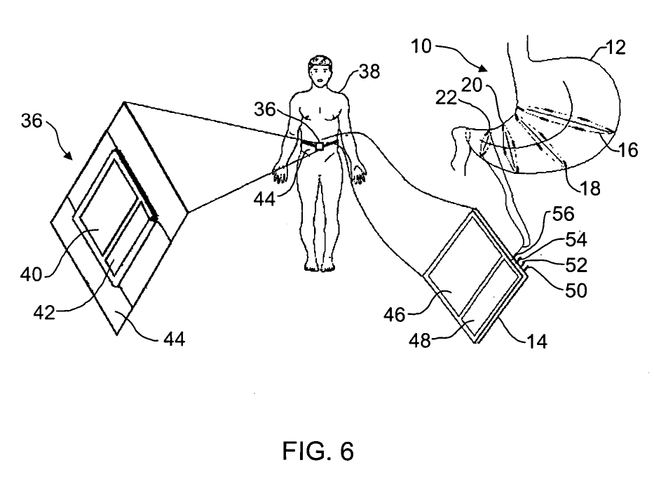

Referring to FIG. 6, in one embodiment an implantable microelectronic apparatus 14 delivers electrical stimulation simultaneously to two electrode sets 16 and 18, and subsequently to two other electrode sets 20 and 22, so that the overall volume reduction of the GI tract 10 is maintained approximately constant over time. In one embodiment the electrical signals are sufficient to produce local circumferential contractions in the vicinity of the electrode sets 16 through 22 and are appropriately timed so that either the proximal sets of circumferential electrodes 16 and 18 or the distal sets 20 and 22 are activated. The microsystem apparatus 14 repeatedly provides electrical stimulation signals to alternating electrode set groups, so that the overall effect is a stable reduction of the gastric volume of the targeted part of the GI tract as a function of time.

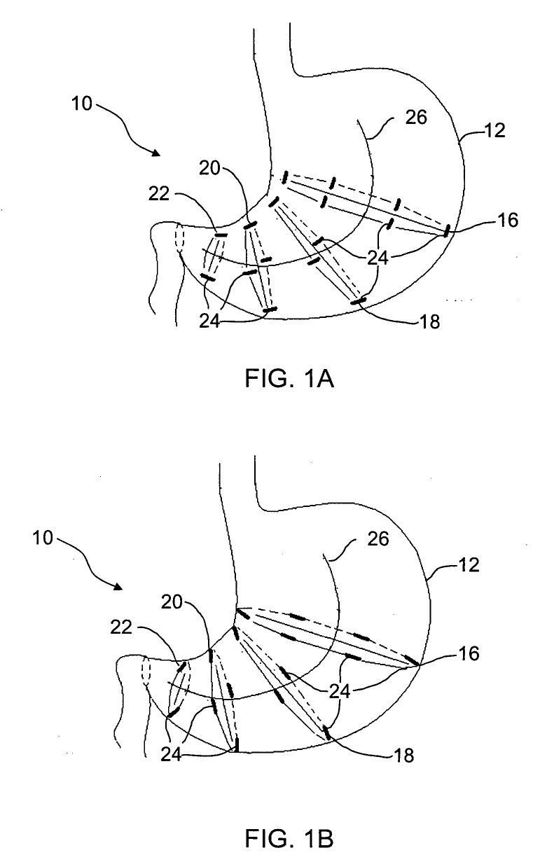

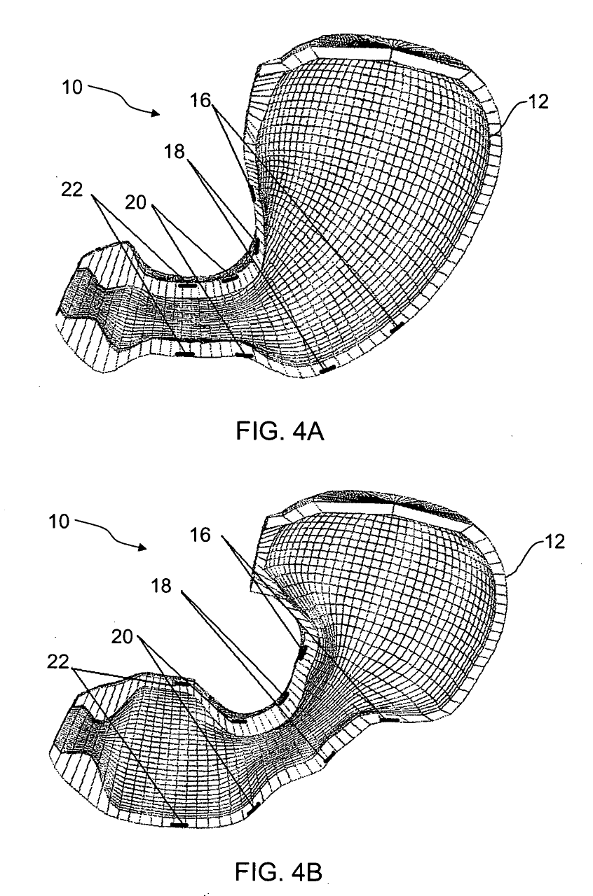

The electrodes 24 are implanted either from the serosal side, as shown in FIG. 4, or the mucosal side, as shown in FIG. 6, of the particular gastrointestinal organ under stimulation. The axes of the electrodes are either collinear to the GI tract axis 26 as shown in FIG. 1A, or perpendicular, to the GI tract axis 26 as shown in FIG. 1B. In some embodiments the electrodes 24 are implanted in pairs, with each electrode set 16 through 22 having two pairs of electrodes 24, However, the number of electrode pairs depends on the circumference of the portion of the GI tract where the electrodes are implanted, and may include as few as one set.

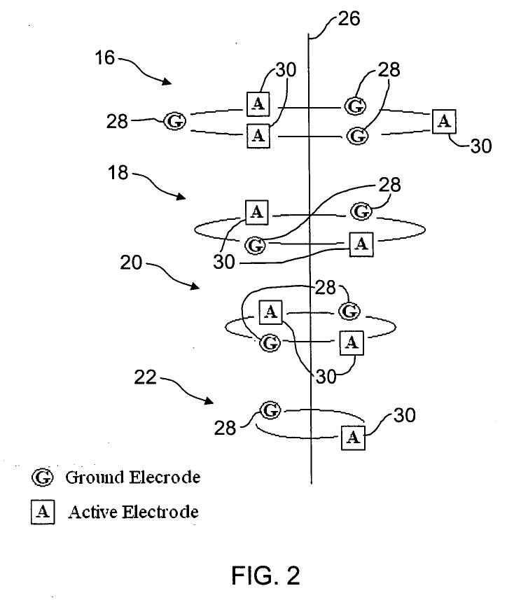

Referring to FIG. 2, in one embodiment each pair of electrodes 24 consists of a ground (reference) 28 electrode and an active electrode 30. In some embodiments local electrode sets 16, 18, 20 or 22 are oriented to correspond to an imaginary line perpendicular to the organ axis 26 in a circumferential (lumen-encompassing) fashion. The number of electrodes 24 in a given set may be calculated knowing the circumference of the lumen of the targeted portion of the GI tract, and assuming that the interelectrode distance should be between 2 and 4 cm, as discussed in Mintchev M. Bowes K, “Computer model of gastric electrical stimulation”, Annals of Biomedical Engineering, 25(4):726-30, 1997 July-August, the content of which is incorporated by reference as if set forth in full.

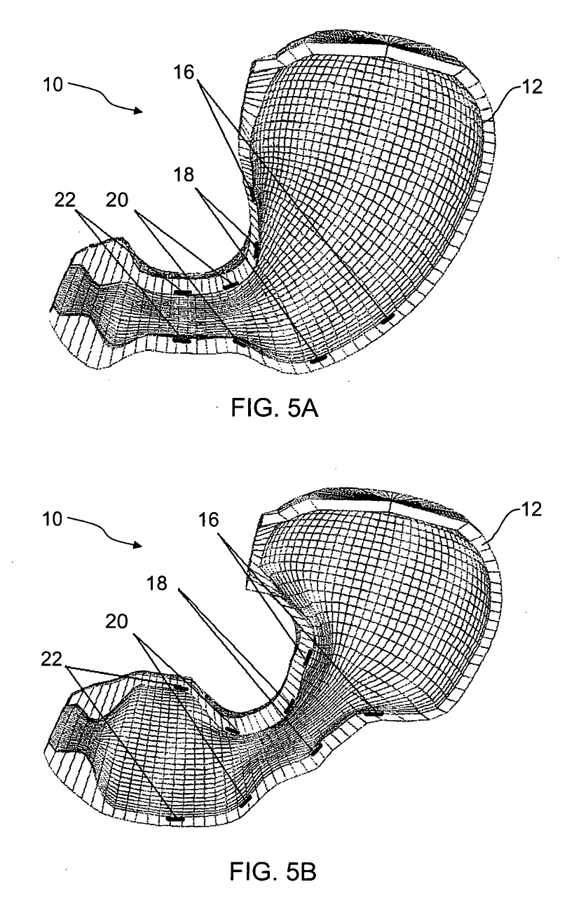

Furthermore, within each of the electrode sets 16 through 22, the electrodes 24 alternate between an active electrode 30 and a reference electrode 28. In some embodiments all active electrodes 30 of a given electrode set are simultaneously exposed to the same electric stimulation signals to produce simultaneous adjacent contractions. The number of electrode sets is determined by the size or dimensions, and particularly by the length of the portion of the GI tract to be stimulated. In some embodiments, when more than two electrode sets are used, an even number of electrode sets is preferred, so that the total number of electrode sets may be divided by two, with the lower half of the sets referred to as “distal” and the upper half of sets referred to as “proximal”. As shown in FIGS. 1, 2, 4 and 5, the electrode sets 16 and 18 are the proximal sets, and the electrode sets 20 and 22 are the distal sets. In the preferred embodiment the stimulation signal is applied at two or more distal and two or more proximal locations.

External Electrical Signals Producing Invoked Peristalsis

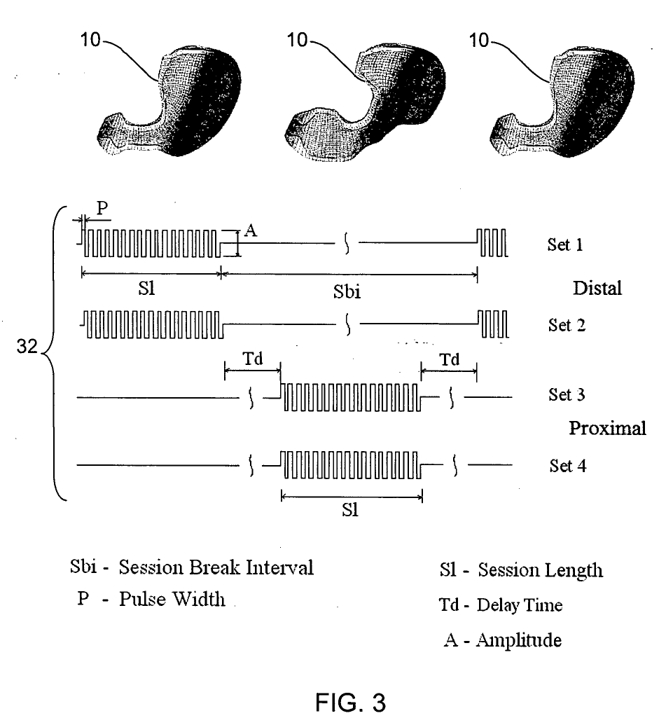

Referring now to FIG. 3, the external electrical signals 32 supplied to the electrode sets, although synchronized between themselves, are applied asynchronously with the spontaneously existing intrinsic slow waves in the GI tract 10, and override the intrinsic slow waves rather than stimulating or enhancing them. FIG. 3 graphically illustrates the relationship between synchronized electrical signals 32 applied to a first anf second set of electrodes located on a distal portion of the stomach and a third and fourth set of electrodes located on a proximal portion of the stomach to the contractile activity induced in the stomach in response to the electrical stimulation signals.

One of skill in the art will appreciate that the present invention is not limited to a particular set of stimulation parameters. Rather the stimulation parameters may be modified as necessary to provide a desired reduction in gastric volume. For example, the applied stimulation energy may be modified to control the duration of the induced contractions to modify the gastric volume as a function of time. More specifically, the strength and duration of induced contractions are related to the parameters of the stimulating pulses, namely the magnitude of the pulses, the pulse frequency, duty cycle and the on time of the train of pulses and the off time between pulse trains.

Further, in one embodiment the stimulation parameters may be varied in response to sensor feedback signals (i.e. force transducers, electrode sensors or the like) to control the strength and duration of the induced contraction. In this embodiment the implantable pulse generator measures the duration of the contraction induced in response to a first set of stimulation parameters and automatically adjusts one or more of the pacing parameters to provide a desired strength/duration of contraction to achieve a desired reduction in gastric volume. In addition, the implantable pulse generator may again utilize feedback signals measured by one or more sensors to adjust any of the stimulation parameters during a particular meal or between meals throughout the day or throughout the course of a particular treatment.

For example, in some embodiments, the frequency (F) of the synchronized electrical signals range from 5 to 50,000 Hz and the amplitudes (A) of the electrical signals range from 3 V peak-to-peak to 30 V peak-to-peak. In some embodiments, the stimulation current may be between 3 mA peak-to-peak and 30 mA peak-to-peak, and may be constant during the stimulation period. In one embodiment the microsystem apparatus delivers electrical stimulation signals simultaneously to two or more electrode sets in bursts of at least one second, and for example between about 10 to 30 seconds duration, represented by the session length (SI) in the representative stimulation parameter set illustrated in FIG. 3. In some embodiments the microsystem apparatus 14 also varies the delay time (Td), which is the time interval between the end of distal stimulation burst and the beginning of the proximal stimulation burst. In some embodiments the delay may be greater than one second, and may not be a constant. Further, in one embodiment the pause between the bursts delivered to a particular electrode set (i.e. the session break interval (Sbi)) equals Td (before the session)+SK+Td (after the session), and may for example fluctuate from 10 to 50 sec. Multiple sessions are administered.

The electrical signal 32 applied to the distal channels, corresponding to sets 20 and 22, and proximal channels, corresponding to sets 16 and 18 (see FIG. 6), may be either direct or alternating. In addition, the electrical stimulation signals 32 may be bipolar or monopolar. In some embodiments the duty cycle of one period of the signal (i.e. whether the pulse duration P is equal to ½ of the period or is smaller) can vary from 10% to 100%, and in one particular embodiment it is 100% (P is equal to one half of the period of the signal). Finally, the shape of the signal can be different (e.g. sinusoidal, triangular, trapezoid, etc.), but in one embodiment it is square or rectangular.

The current delivery capability of the microsystem can be estimated considering the average total current consumption per unit muscular thickness of GI tissue per electrode pair, which is approximated as 3 mA/mm. With the assumption that the thickness of the muscle is in the range of 2.5 mm to 3.55 mm, the average total current drawn by the tissue is estimated to be in the range of 7.5 mA to 10.5 mA.

Microsystem Device

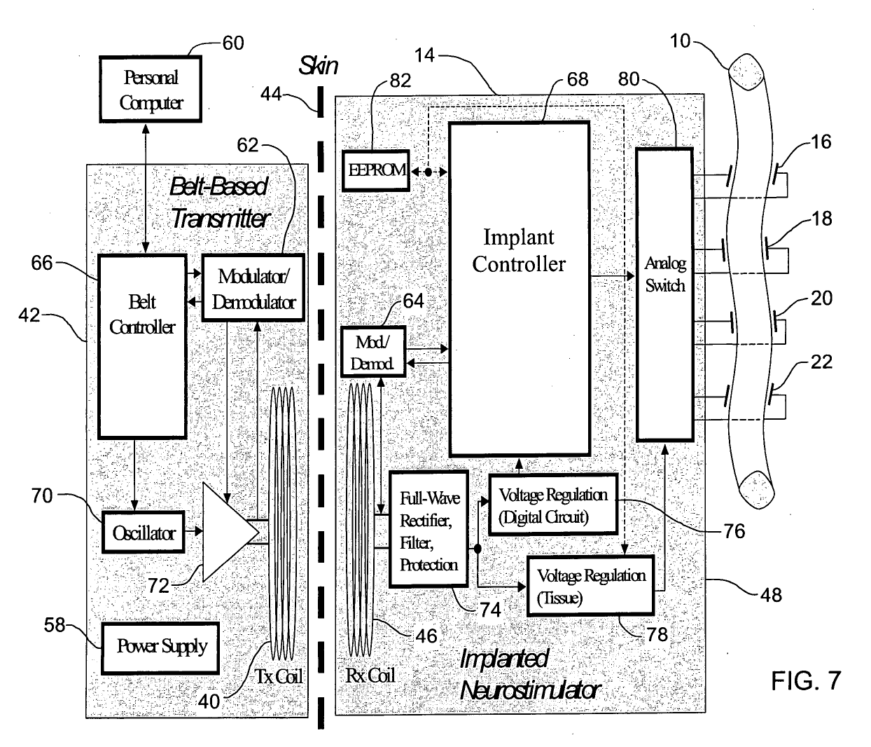

Referring to FIG. 6, the power supply in one embodiment of the implantable microsystem 14 is, by way of example, an autonomous battery (not shown), which in some embodiments can be rechargeable through a transcutaneous inductive link facilitated by an abdominal belt 36 periodically worn by the patient 38, preferably during sleep. As depicted in FIGS. 6 and 7, the power supply may also utilize transcutaneous power transfer facilitated by an abdominal belt 36 worn by the patient 38 during the periods of the desired gastrointestinal organ control. In the distributed microsystem setup shown in FIG. 6, the external control is administered via the abdominal belt 36, in which the transmitting inductive coil 40 for transcutaneous power transfer is positioned along with the associated microcontroller-based electronics 42. The microcontroller-based electronics for one embodiment are shown in greater detail in FIG. 9.

In some embodiments the belt is attached to the body in the abdominal area 44. The implanted microsystem 14 is implanted on the inner side of the abdominal wall right under the abdominal bell center. In these embodiments, the implanted microsystem contains a receiving coil 46 which is aligned with a transmitting coil 40 in the belt and microcontroller-based electronics 48. In the case of an autonomous non-rechargeable battery-based power supply for the implanted microsystem, transmitting and receiving coils are not necessary and the dimensions of both microsystems can therefore be reduced. The implanted microsystem is shown with four channels 50, 52, 54, and 56. Channel 56 is shown to be connected to set 22, and the other channels will also be connected to the corresponding sets of electrodes.

FIG. 7 depicts a simplified block-diagram of the system. One of skill in the art will appreciate that Very-Large-Scale-Integration (VLSI) of the stimulation system may be utilized if further device miniaturization is desired. In this particular implementation the battery of the implantable device can be autonomous or externally rechargeable. The communication between the controlling microsystem 42 located in the abdominal belt 36 and the implant 14 is provided by modulating the wireless power supply passing between transmitting coil 40 and receiving coil 46 using modulator/demodulators 62 and 64, which are controlled by the belt controller 66 and the implant controller 68, respectively.

Once the implant is in place, external control circuitry 60, such as a personal computer, for example, can be utilized to control the stimulation parameters, the number of stimulation sessions and the pause between successive sessions. As shown in FIG. 7, the belt controller 66, which in one embodiment is controlled by the personal computer 60, causes a signal to be sent through transmitting coil 40 using oscillator 70 and amplifier 72. This signal is received by receiving coil 46, and rectified into a DC power source by rectifier 74. The resulting DC power source is used to power the implant controller 68 through voltage regulator 76, and to stimulate the target GI tract tissue 10 through electrode sets 16, 18, 20 and 22 through a voltage regulator 78.

In some embodiments the implant controller 68 controls each electrode set 16, 18, 20 and 22 individually or in the alternative as groups according to their position through the analog switch 80, which allows the waveforms of the electrical signals 32 to be controlled, for example, similar to those shown in FIG. 3. A memory such as, by way of example, an EEPROM 82 is also included to act as a memory source for the implant controller 68. It will be understood by those skilled in the art that the design of the electronic controllers 42 and 48 is exemplary, and that other configurations are possible to yield similar results that fall within the scope of the invention.

Immaterial modifications may be made to the embodiments of the invention described here without departing from the invention.

We claim:

1. A method of reducing gastric volume in a portion of a gastrointestinal tract having a longitudinal axis, the method comprising repeating a stimulation cycle comprising:
stimulating at least a first contraction at a first location on the gastrointestinal tract for a first stimulation period; and
stimulating at least a second contraction at a second location on the gastrointestinal tract for a second stimulation period;
wherein the first and second locations are axially spaced from each other along the longitudinal axis, and in which the first and second stimulation periods are sufficient to maintain a gastric volume reduction in the portion of the gastrointestinal tract at least during the stimulation cycle.

2. The method of claim 1 in which the portion of the gastrointestinal tract is the stomach.

3. The method of claim 1 in which stimulating the first contraction and the second contraction is carried out using respective first and second sets of electrodes implanted on the stomach.

4. The method of claim 3 in which the electrodes of each of the first and second sets of electrodes are spaced circumferentially around the stomach.

5. The method of claim 4 in which each of the first and second sets of electrodes comprises electrodes at different positions along the gastric axis.

6. The method of claim 5 in which each of the first and second sets of electrodes comprises at least a pair of electrode sub-sets, in which the electrodes are spaced around the circumference of the portion of the gastrointestinal tract being stimulated.

7. The method of claim 3 in which the electrodes of the first and second sets of electrodes are implanted sub-serosally or sub-mucosally.

8. The method of claim 1 in which the stimulation cycle comprises applying stimulating energy at a frequency between 5 to 50,000 Hz.

9. The method of claim 1 in which the stimulation cycle comprises applying stimulating energy having an amplitude between 3 V peak-to-peak and 30 V peak-to-peak.

10. The method of claim 1 in which the stimulation cycle comprises applying stimulating energy in sessions of pulses, wherein each session has a duration of at least 1 second.

11. The method of claim 1 in which contractions of consecutive stimulation cycles at a location are separated by a greater time period than the length of the stimulation periods of the respective contractions at the location.

12. The method of claim 11 in which contractions of consecutive stimulation cycles at one of the first location and the second location are separated by an equal or greater time period than the length of the stimulation periods of the respective contractions at the other of the first location and the second location.

13. The method of claim 1 in which there is a delay between the end of the first stimulation period and the beginning of the first stimulation period, and the delay varies between different stimulation sessions.

14. The method of claim 1 in which:
stimulating at least a first contraction comprises applying electrical stimulation to neural pathways at the first location of the gastrointestinal tract; and
stimulating at least a second contraction comprises applying electrical stimulation to neural pathways at the second location of the gastrointestinal tract.

15. The method of claim 14 in which:
the neural pathways at the first location have a first exhaustion period;
the neural pathways at the second location have a second exhaustion period;
the first stimulation period is less than or equal to the first exhaustion period in any one stimulation cycle; and
the second stimulation period is less than or equal to the second exhaustion period in any one stimulation cycle.

16. The method of claim 1 in which the stimulation cycle further comprises:
stimulating a contraction at a third location on the gastrointestinal tract for a third stimulation period;
the first, second and third locations being axially spaced from each other along the gastric axis.

17. The method of claim 1 in which the stimulation cycle causes an overall contraction of the portion of the gastrointestinal tract stimulated, and the overall contraction is maintained approximately constant during a number of stimulation cycles.

18. The method of claim 3 in which electrodes in the first and second set of circumferentially spaced electrodes have alternating polarity.

19. The method of claim 4 in which electrodes in the first and second set of circumferentially spaced electrodes are 2-4 cm apart measured circumferentially.

20. The method of claim 1 in which stimulating at least the first contraction and the second contraction is carried out asynchronously with spontaneously existing slow waves in the first location and the second location.

21. The method of claim 3 in which the stimulation cycle comprises applying excitation energy to the electrodes with a duty cycle having a pulse duration less than or equal to one half of the duty cycle.

22. A method of causing muscles to contract in a portion of the gastrointestinal tract having a longitudinal axis, the method comprising the steps of:
implanting a set of electrodes on the portion of the gastrointestinal tract, the set of electrodes being implanted to form a pair of circumferentially spaced electrode sub-sets; and
energizing electrodes in the set of electrodes simultaneously to cause a contraction of smooth muscle of the portion of the gastrointestinal tract by signals applied via neural pathways in the vicinity of the electrodes, the signals being applied for a duration sufficient to maintain a gastric volume reduction in the portion of the gastrointestinal tract.

23. The method of claim 22, further comprising the steps of:
implanting a second set of electrodes on a second portion of the gastrointestinal tract, the second set of electrodes being implanted to form a pair of circumferentially spaced electrode sub-sets; and
energizing electrodes in the second set of electrodes simultaneously to cause a contraction of smooth muscle of the portion of the gastrointestinal tract by signals applied via neural pathways in the vicinity of the electrodes, the signals being applied for a duration sufficient to maintain a gastric volume reduction in the portion of the gastrointestinal tract, wherein the second set of electrodes are not energized while the first set of electrodes are energized.
